(19) United States
(12) Patent Application Publication
(10) Pub. No.: US 2019/0388044 A1
(43) Pub. Date: Dec. 26, 2019

(54) RADIATION FLUOROSCOPIC IMAGING APPARATUS

(71) Applicant: SHIMADZU CORPORATION, KYOTO-SHI (JP)

(72) Inventors: DAI HIROSE, KYOTO-SHI (JP); Koki YOSHIDA, KYOTO-SHI (JP)

(73) Assignee: SHIMADZU CORPORATION, KYOTO-SHI (JP)

(21) Appl. No.: 16/017,485

(22) Filed: Jun. 25, 2018

(51) Int. Cl. A61B 6/00; A61B 6/04

(52) U.S. Cl. CPC A61B 6/487; A61B 6/0407; A61B 6/4464; A61B 6/4441; A61B 6/0457

(57) ABSTRACT

An X-ray imaging apparatus achieves X-ray fluoroscopy and X-ray imaging in the larger range by combining a sliding action or rotation of an arcuate C-arm and an tilting-and-rolling of the table in combination. As noted while the C-arm suspends sliding after the C-arm slides in the sagittal direction, the table of the examination table tilts. Then, an angle θ between the axis line from the X-ray irradiation element 31 to the flat panel detector 32 and the surface of the table 11 coincides with the target angle at which the X-ray fluoroscopy and the X-ray imaging is carried out. Then after, the table moves in the longitudinal direction and lowers.

### CROSS REFERENCE TO RELATED APPLICATIONS

This application relates to, but does not claim priority from, JP 2015-255996 filed Dec. 28, 2015, published as JP 2017-118910 on Jul. 6, 2017, the entire contents of which are incorporated herein by reference.

### FIGURE SELECTED FOR PUBLICATION

FIG. 8

### BACKGROUND OF THE INVENTION

### Field of the Invention

The present invention relates to a radiation fluoroscopy imaging apparatus comprising a C-arm (having an arcuate C-shape) that holds a radiation irradiation element and a radiation detector while facing each other and performs a radiation fluoroscopy and a radiation imaging of a subject.

### Description of the Related Art

An X-ray fluoroscopy imaging apparatus, for example used for a cardiovascular examination and surgery, comprises an examination table on which a subject is loaded, an X-ray irradiation element having an X-ray tube, an X-ray detector that detects the X-ray irradiated from the X-ray tube and transmitting through the subject on the table, the C-arm that has a circular (arch-like) guide element and an approximately letter C-like appearance and supports the X-ray tube and the X-ray detector, a sliding mechanism that supports slidably the C-arm by connecting with the circular (arch) guide element and a rotation mechanism that supports the C-arm rotatably around the axis facing the horizontal direction via the sliding mechanism.

According to such X-ray fluoroscopy imaging apparatus, relative to the oblique direction movement of the C-arm around the body axis of the subject, the left movement direction from the head of the subject denotes the left anterior oblique (LAO) and the right movement direction therefrom denotes the right anterior oblique (RAO). Relative to the sagittal direction movement of the C-arm along the body axis of the subject in the body axis direction, the movement direction toward the head of the subject denotes CRAN (cranial) and the movement direction toward the foot thereof denotes CAUD (caudal).

According to such X-ray fluoroscopy imaging apparatus, when the C-arm slides in the sagittal direction and the sliding amount of the C-arm is large, the table, the subject on the table or the other instrumentation arranged in the examination room interfere with the X-ray irradiation element or the X-ray detector supported by the C-arm, so that the sliding amount of the C-arm is restricted. In addition, when the C-arm slides in the sagittal direction or the oblique direction, the sliding amount (range) of the C-arm is restricted within the predetermined range.

The below-noted Patent Document 1, the entire contents of which are incorporated herein by reference, discloses an X-ray imaging apparatus comprising; an image (external profile) data registration means that registers the three-dimensional model external profile data corresponding to the three-dimensional external structure of the X-ray imaging mechanism and the three-dimensional model external profile data corresponding to the external structure of the table; a relative location relationship extraction means between the X-ray imaging mechanism and the table, which carries out a real-time extraction based on the present location of the X-ray imaging apparatus and the three dimensional model external profile data and the location of the table and the three dimensional model external profile data; an imaging system movement control means that controls the X-ray imaging system moving mechanism while considering the relative location relationship information between the X-ray imaging mechanism and the table, which is extracted by the locational relationship extraction means.

According to such as an X-ray imaging apparatus, the present state with regard to the facing condition between the external surfaces of the X-ray imaging mechanism and the table, in which the X-ray imaging mechanism may contact the table, can be reflected on the movement of the X-ray imaging mechanism by the X-ray imaging system moving mechanism with no difficulty and a complete accuracy.

### RELATED PRIOR ART DOCUMENTS

### Patent Document

Patent Document 1: Patent Published JP2004-255075

### ASPECTS AND SUMMARY OF THE INVENTION

### Objects to be Solved

With regard to sliding the arcuate type C-arm, an operator may operate a lever to slide an arcuate-type C-arm (the first method) or may slide the C-arm to the location corresponding to the three-dimensional image by interlocking operation of the three-dimensional image by rotating the three-dimensional image denoting the C-arm (the second method). In the first method, when the sliding amount of the C-arm is out of (over) the restriction range therefor, the C-arm suspends at the instant location regardless the lever operation by the operator. In the second method, the sliding amount relative to the three-dimensional image is out of the restriction range of the sliding amount of the C-arm, so that the sliding of the C-arm is not carried out.

Even in such a case, however, the X-ray fluoroscopy or the X-ray imaging may be accomplished in the wider angle range out of the restriction range of the sliding amount for the C-arm by tilting-and-rolling the table of the examination table, on which the subject is loaded. The same phenomenon may take place in the case of moving the X-ray irradiation element and the X-ray detector by rotating the C-arm instead of moving the X-ray irradiation element and the X-ray detector by sliding the C-arm.

The purpose of the present invention is to solve the above objects and to provide an X-ray imaging apparatus that achieves X-ray fluoroscopy and X-ray imaging in the larger range by combining a sliding action or rotation of the C-arm and tilting-and-rolling of the table.

### Means for Solving the Problem

A radiation imaging apparatus, according to one aspect of the present invention claimed in claims, comprises an examination table comprising a table on which a subject is laid and a table tilting-and-rolling mechanism that carries out tilting and rolling the table; and a radiation imaging element comprising a radiation irradiation element, a radiation detector that detects the radiation that is irradiated from the radiation irradiation element and transmits through the subject on the table, a C-arm that supports the radiation irradiation element and the radiation detector while facing each other, and a C-arm moving mechanism that supports the C-arm slidably and rotatably, wherein the table tilting-and-rolling mechanism carries out tilting and rolling the table when a target angle to achieve radiation fluoroscopy or radiation imaging is out of a range of an imaging angle due to sliding or rotating the C-arm, so that the imaging angle coincides with the target angle.

According to another aspect of the present invention claimed in claims, the examination table further comprises a table moving mechanism that moves the table in the longitudinal direction of the table, the width (lateral) direction of the table and the vertical direction, wherein the table is moved in any direction including the longitudinal direction of the table, the width (lateral) direction of the table and the vertical direction so that the radiation from the radiation irradiation element toward the radiation detector passes through the same location of the subject, or the distance between subject and the radiation irradiation element or the radiation detector is constant when the table moving mechanism tilts or rolls the table.

According to another aspect of the present invention claimed in claims, the table tilting-and-rolling mechanism tilts the table and the table moving mechanism moves the table in the longitudinal direction and the vertical direction when the radiation irradiation element and the radiation detector slide in the sagittal direction relative to the subject.

According to another aspect of the present invention claimed in claims, the table tilting-and-rolling mechanism rolls the table and the table moving mechanism moves the table in the width direction and the vertical direction when the radiation irradiation element and the radiation detector slide in the oblique direction relative to the subject.

According to another aspect of the present invention claimed in claims, the table tilts and rolls following sliding the C-arm near by the sliding range limit thereof or rotating the C-arm near by the rotation limit thereof when the C-arm is operative to slide or rotate.

According to another aspect of the present invention claimed in claims, the radiation imaging apparatus further comprises an input element that is operative to input the target angle, and a calculation element that calculates the required angle of tilting and rolling of the table from the target angle and the imaging angle based on the slide or rotation of the C-arm, wherein the table tilting-and-rolling mechanism tilts and rolls the table based on the operation (calculation) results by the calculation element.

### Effect of the Invention

According to one aspect of the present invention claimed in claims, the C-arm is operative in a larger angle range to execute the radiation fluoroscopy or the radiation imaging by combining slide or rotation of the C-arm and tilting-and-rolling of the table, compared to the case in which only either one of slide and rotation of the C-arm is executed.

According to the other aspects of the present invention claimed in claims, even when the table tilts and rolls, the X-ray fluoroscopy or the X-ray imaging is operative to perform on the subject relative to the same location thereof.

According to another aspect of the present invention claimed in claims, sliding or rotation of the C-arm and tilting or rolling of the table are operative to be continuously executed by the same operation as the normal sliding operation or the normal rotation operation.

According to another aspect of the present invention claimed in claims, sliding or rotation of the C-arm and tilting or rolling of the table are operative to be executed automatically by inputting a target angle.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a perspective front view illustrating an X-ray fluoroscopy imaging apparatus according to an aspect of the present invention.

FIG. 2 is a perspective side view illustrating an X-ray fluoroscopy imaging apparatus according to the aspect of the present invention.

FIG. 3 is a perspective oblique back view illustrating an X-ray fluoroscopy imaging apparatus according to the aspect of the present invention.

FIG. 4 is a schematic view illustrating a table moving mechanism relative to an examination table 1.

FIG. 5 is a block diagram illustrating the principal control system of the X-ray fluoroscopy imaging apparatus according to an aspect of the present invention.

FIG. 6 is a schematic view illustrating the sliding action of the C-arm 28 and the tilting-and-rolling action of the table 11.

FIG. 7 is a schematic view illustrating the sliding action of the C-arm 28 and the tilting-and-rolling action of the table 11.

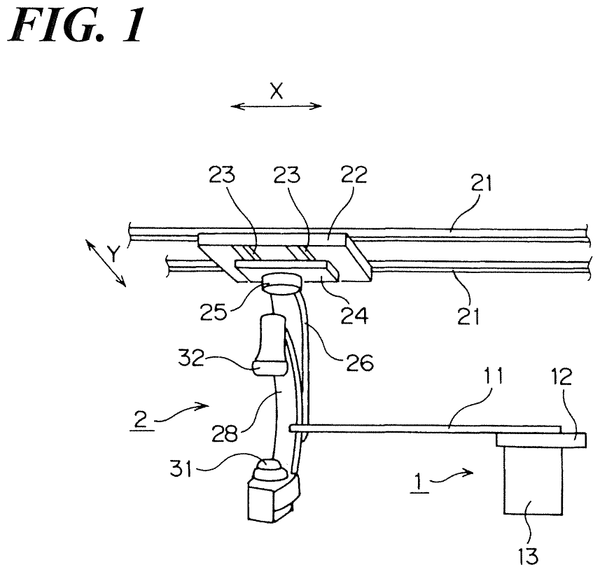

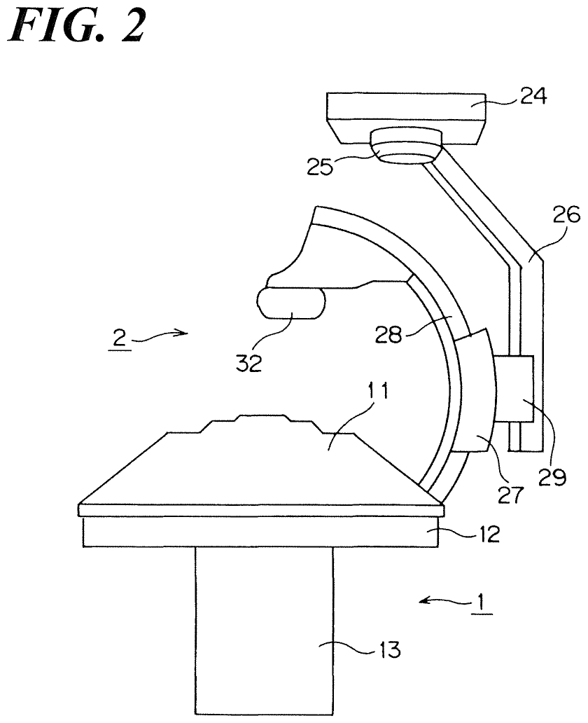

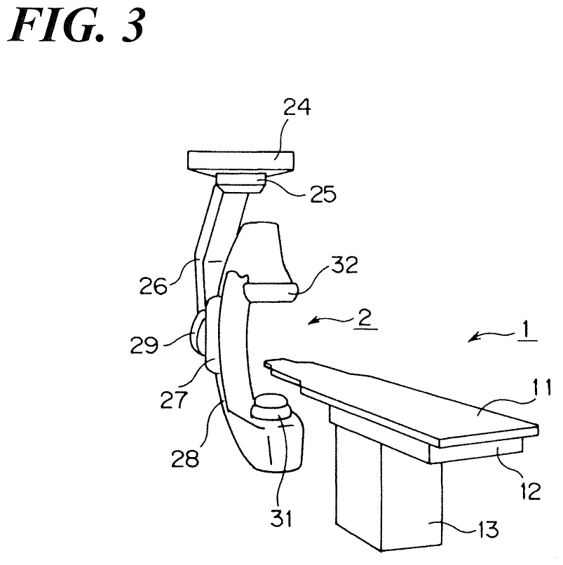

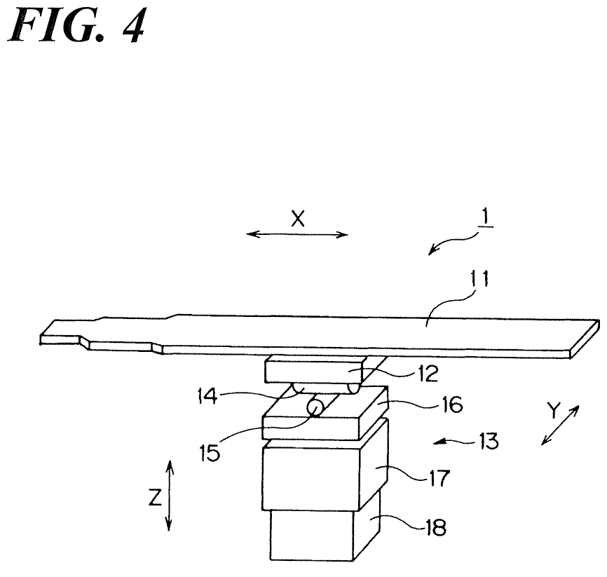

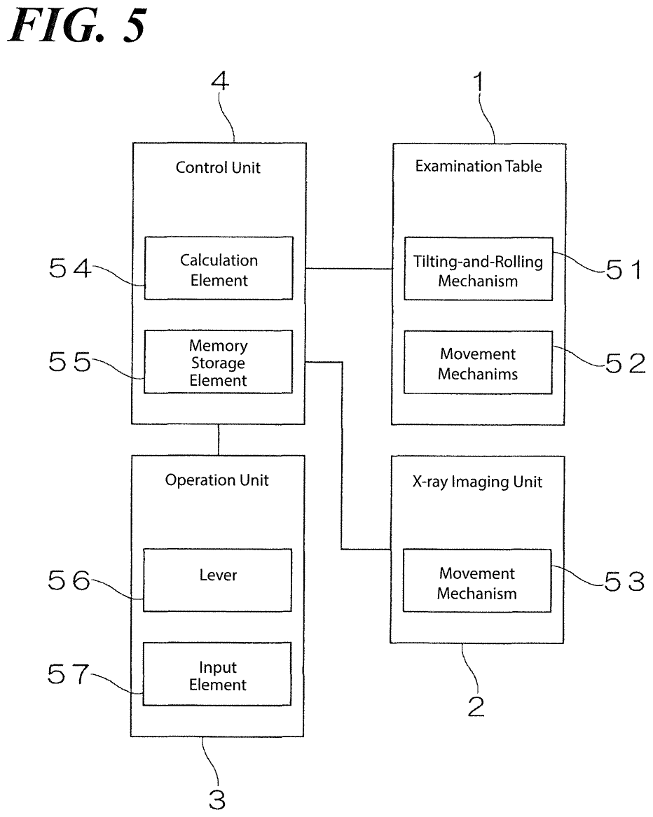

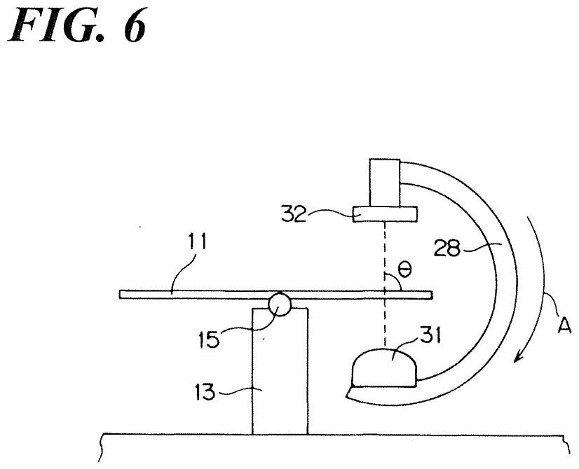

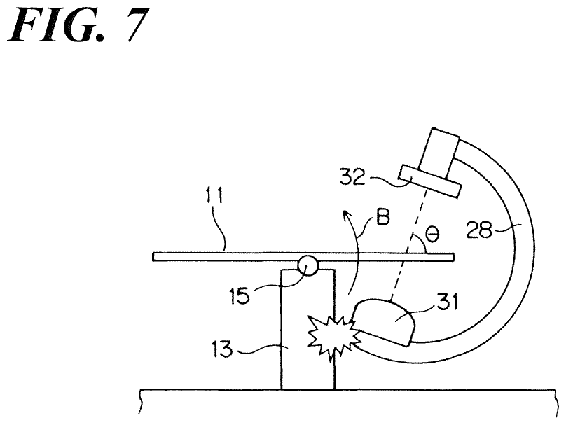

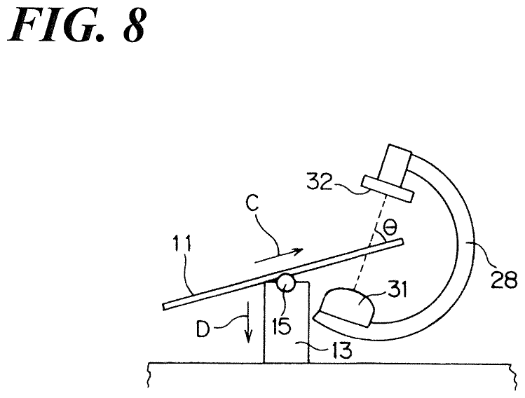

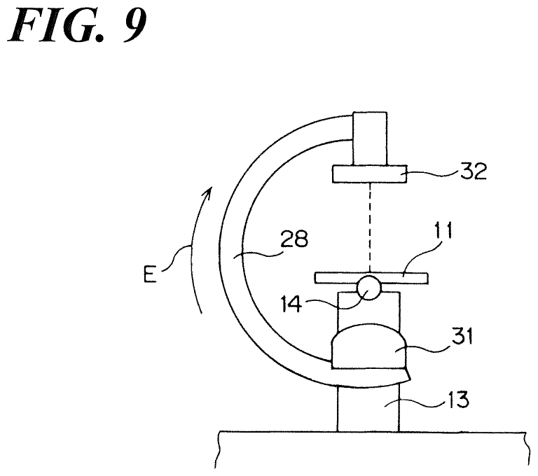

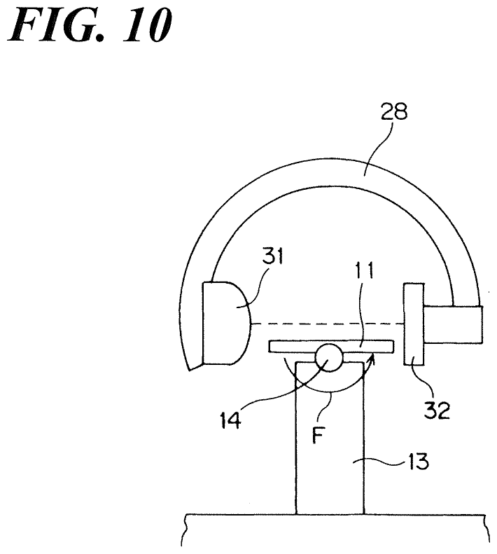

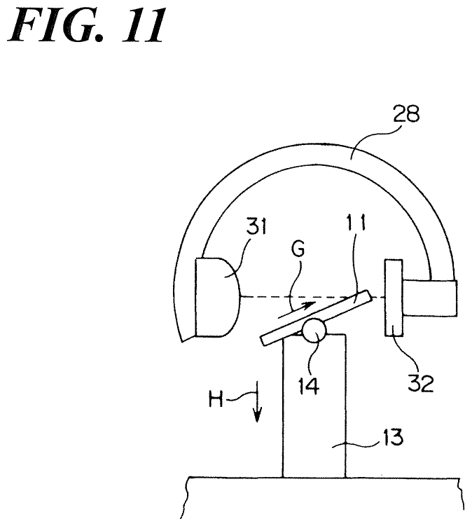

FIG. 8 is a schematic view illustrating the sliding action of the C-arm 28 and the tilting-and-rolling action of the table 11.

FIG. 9 is a schematic view illustrating the sliding action of the C-arm 28 and the tilting-and-rolling action of the table 11.

FIG. 10 is a schematic view illustrating the sliding action of the C-arm 28 and the tilting-and-rolling action of the table 11.

FIG. 11 is a schematic view illustrating the sliding action of the C-arm 28 and the tilting-and-rolling action of the table 11.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

As used herein, a ‘computer-related’ or ‘computer-related’ type systems involve an input device, of any kind for receiving data of any kind, an output device, of any kind, for outputting data of any kind, in tangible form (e.g., electronic data points, image pixel display data, print or display data, measurement data in any form stored in a transitory memory or permanent memory for storing data as well as computer code, and a processor, controller, or operative element for executing program instruction and communications received via said input device, process said data within said microprocessor and output said processed data via said output device.

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related radiation fluoroscopic imaging apparatus devices, computers, processors, systems, and operational controls and technologies of radiographic devices and all their sub components, including various circuits and combinations of circuits without departing from the scope and spirit of the present invention.

FIG. 1 is a perspective front view illustrating an X-ray fluoroscopy imaging apparatus according to the aspect of the present invention. FIG. 2 is a perspective side view illustrating an X-ray fluoroscopy imaging apparatus according to the aspect of the present invention. FIG. 3 is a perspective oblique back view illustrating an X-ray fluoroscopy imaging apparatus according to the aspect of the present invention. In addition, FIG. 1, FIG. 2, are illustrating that the C-arm 28 slides in the oblique direction and FIG. 3 is illustrating that the C-arm 28 slides in the sagittal direction.

The X-ray fluoroscopy imaging apparatus of the present invention comprises an examination table 1 and an X-ray imaging element 2.

The X-ray imaging unit 2 further comprises a radiation irradiation element 31 comprising an X-ray tube and a collimator of the present invention, a flat panel detector 32 as an X-ray detector that detects the X-ray which is irradiated from the X-ray irradiation element 31 and transmits through the irradiated subject on the examination table 1. In addition, the X-ray imaging unit 2 further comprises a C-arm 28, having approximately a letter C shape, that comprises a circular guide and supports the X-ray irradiation element 31 and the flat panel detector 32 while facing each other, a slide mechanism 27 that supports the C-arm 28 to be slidable by connecting with the guide of the C-arm 28, a rotation mechanism 29 that supports the slide mechanism 27 to be rotatable, and a support member 26 that supports the rotation mechanism 29.

The support member 26 is supported through the rotation member 25 that rotates around the vertical axis relative to the first movement member 24. The first movement member 24 is movable along a pair of rails 23, which is installed underneath the second movement member 22, in the Y-direction referring to FIG. 1. In addition, the first movement member 24 is movable along a pair of rails 21, which is installed on the ceiling of the examination room, in the X-direction referring to FIG. 1.

Therefore, the C-arm 28 moves along with the X-ray irradiation element 31 and the flat panel detector 32 in the X-direction and Y-direction and rotates around the vertical axis, so that the C-arm 28 is operative to move between the location indicated in FIG. 1, FIG. 2 and the location indicated in FIG. 3, rotate around the rotation member 29, and slide relative to the slide member 27.

FIG. 4 is a schematic view illustrating a table moving mechanism relative to an examination table 1. In addition, FIG. 4 is illustrating the inside structure of a pedestal 13 in FIG. 1-FIG. 3.

Referring to FIG. 1-FIG. 4, the examination table 1 comprises the table 11, on which the subject is loaded, a frame 12 and the pedestal 13.

The table 11 is supported to be movable in the longitudinal direction of the table 11 (X-direction indicated in FIG. 4 in the state in which the table 11 is in-place in the horizontal direction) relative to the frame 12. In addition, the frame 12 oscillates around both the axis 14 facing the width (lateral) direction of the table 11 (Y-direction indicated in FIG. 4 in the state in which the table 11 is in-place in the horizontal direction) and the axis 15 facing the longitudinal direction. Therefore, the table 11 is operative to achieve a roll oscillating around the axis 14 and a tilt oscillating around the axis 15 along with the frame 12.

The X-ray irradiation element 14 and the X-ray detection element 15 are supported by a movement table 16. Such a movement table 16 is movable in the width direction of the table 11 relative to the lifting pedestal (column) 17. In addition, the lifting column 17 is movable in the vertical direction (Z-direction indicated in FIG. 4) relative to the pedestal (support) base 18. In addition, as set forth above, the table 11 is supported to be movable in the longitudinal direction of the table 11 relative to the frame 12. Therefore, the table 11 is movable in both the longitudinal direction thereof and the width direction thereof and in addition, liftable in the vertical direction.

FIG. 5 is a block diagram illustrating the principal control system of the X-ray fluoroscopy imaging apparatus according to the aspect of the present invention.

Such an X-ray fluoroscopy apparatus comprises a ROM that stores operation programs required to control the apparatus, a RAM that stores the data temporally and so forth when controlling, a CPU that executes the logic operation, and so forth and further comprises a control unit 4 that controls the entire apparatus. Such a control unit 4 comprises a calculation element 54 that calculates an angle with which the table 11 achieves at least one of tilting and rolling so that the imaging angle coincides with the target angle when the target angle to carries out X-ray fluoroscopy or X-ray imaging is out of the range of the imaging angle due to the slide of the C-arm 28, and a memory storage element 55 that stores the locational information including dimension of each element of the examination table 1 and the X-ray imaging unit 2 and positioning thereof.

Here, the target angle and the imaging angle is defined based on the relative location between the table 11 and the C-arm 28. In addition, as set forth later, the imaging angle includes a movable angle of the C-arm 28 per se and a movable angle restricted by interference between the X-ray irradiation element 31 and the flat panel detector 32 and other instrumentations in the examination room other than an angle restricted by interference between the X-ray irradiation element 31 and the flat panel detector 32 and other members such as the table 11.

The control unit 4 connects with the operation unit 3 to operate the examination table 1 and the X-ray imaging unit 2. In addition, the control unit 4 that connects with the examination table 1 controls the tilting-and-rolling mechanism 51 that tilts and, rolls the table 11 of the examination table 1 and the movement mechanism 52 that moves the table 11 in the longitudinal direction and the width direction and in addition, moves up-and-down (vertically) the table 11.

As follows, the inventors set forth the sliding action of the C-arm 28 and the tilting-and-rolling action of the table 11 when the X-ray fluoroscopy imaging apparatus having the above structure carries out X-ray fluoroscopy or X-ray imaging. FIG. 6-FIG. 8 are schematic views illustrating the sliding action of the C-arm 28 and the tilting-and-rolling action of the table 11. In addition, in the Embodiment referring to FIG. 6-FIG. 8, the C-arm 28 approaches the subject from the head thereof and slide in the sagittal direction.

The C-arm 28 is in-place in the position indicated in FIG. 6 in the default state prior to carrying out X-ray imaging or X-ray fluoroscopy. It is given that the C-arm 28 slides in the sagittal direction indicated by the arrow A in FIG. 6 so that the angle θ between the axis line of X-axis from the X-ray irradiation element 31 to the flat panel detector 32 and the surface of the table 11 is smaller from the above default state. In such a case, the C-arm 28 starts sliding following the operation of the lever 56 of the operation unit 3 by the operator referring to FIG. 5. In such a case, the control unit 4 sends a directive for sliding the C-arm 28 to the movement mechanism 53 of the X-ray imaging unit 2.

Referring to FIG. 7, when the C-arm slides continuously, the tip of the C-arm 28 and the pedestal 13 of the examination table 1 collide with each other. The control unit 4 recognizes such a collision from the locational information stored in the memory storage element 55 and suspends the movement thereof near the limit of the sliding amount before the C-arm 28 contacts the pedestal 13. Then, the control unit 4 sends the directive to the tilting-and-rolling mechanism 51 of the examination table 1 and tilts the table 11 of the examination table 1 toward the direction indicated by the arrow B in FIG. 7.

The operator suspends the operation of the lever 56 of the operation unit 3 when the angle θ between the axis line from the X-ray irradiation element 31 to the flat panel detector 32 and the surface of the table 11 coincides with the target angle at which the X-ray fluoroscopy and the X-ray imaging is carried out or is equivalent thereto according to the tilting action of the table 11. Accordingly, the tilting action of the table 11 suspends. In addition, the equivalent angle is the angle almost the same as the target angle. Hereinafter, such an equivalent angle denotes simply the target angle.

In such a state, the angle θ between the axis line from the X-ray irradiation element 31 to the flat panel detector 32 and the surface of the table 11 coincides with the target angle at which the X-ray fluoroscopy and the X-ray imaging is carried out. However, an imaging positioning for the X-ray fluoroscopy or the X-ray imaging relative to the subject shifts along with the tilting action of the table 11 and in addition, the subject is closer to the flat panel detector 32. Therefore, the table 11 is moved in the longitudinal direction indicated by the arrow C in FIG. 8 and lowered as indicated by the arrow D using the movement mechanism 52 of the examination table 1, so that the X-ray always transmits the same region of the subject and the distance between the subject and the flat panel detector 32 or the X-ray irradiation element 31 is constant.

In addition, in the Embodiment set forth above, when the operator operates the lever 56 of the operation unit 3 and then the angle θ between the axis line from the X-ray irradiation element 31 to the flat panel detector 32 and the surface of the table 11 coincides with the target angle at which the X-ray fluoroscopy and the X-ray imaging is carried out, the slide of the C-arm 28 suspends. On the other hand, when the C-arm 28 slides to the position corresponding to the three-dimensional image by rotating the three-dimensional image denoting the C-arm 28 and using the three-dimensional image interlocking operation, the calculation element 54 of the control unit 4 calculates the sliding amount of the C-arm 28 and the tilting amount of the table 11.

Specifically, when the C-arm 28 slides in such a way, the operator inputs the target angle using the input element 57, such as a keyboard, of the operation unit 3, at which the X-ray fluoroscopy or the X-ray imaging is carried out. Then, the calculation element 54 of the control unit 4 calculates the angle of the tilt of the table 11 along with the slide of the C-arm 28 based on the locational information including such as the dimension and arrangement of each element of the examination table 1 and the X-ray imaging unit 2, which are stored in the memory storage element 55, so that the imaging angle coincides with the target angle. And based on the calculation results, the tilting-and-rolling mechanism 51 of the examination table 1 tilts the table 11 and in addition, the movement mechanism 53 of the X-ray imaging unit 2 slides the C-arm 28.

According to the Embodiment referring to FIG. 6-FIG. 8, the C-arm 28 approaches the subject from the head-side thereof and the C-arm slides, so that the X-ray irradiation element 31 and the flat panel detector 32 move in the sagittal direction relative to the subject. On the other hand, alternatively, the C-arm 28 approaches the subject from the body-side thereof and the C-arm rotates in accordance with actions of the slide member 27 and the rotation member 29, so that the X-ray irradiation element 31 and the flat panel detector 32 move in the sagittal direction relative to the subject.

In such a case, the tilting action and the movement action of the table 11 are the same as the Embodiment set forth referring to FIG. 6-FIG. 8.

As follows, according to the aspect of an alternative Embodiment, the inventors set forth the sliding action of the C-arm 28 and the tilting-and-rolling action of the table 11 when the X-ray fluoroscopy imaging apparatus set forth above carries out X-ray fluoroscopy or X-ray imaging. FIG. 9-FIG. 11 are schematic views illustrating the sliding action of the C-arm 28 and the tilting-and-rolling action of the table 11. In addition, FIG. 9-FIG. 11 are illustrating that the C-arm 28 slides in the oblique direction.

The C-arm 28 is in-place in the position indicated in FIG. 9 in the default state prior to carrying out X-ray imaging or X-ray fluoroscopy. It is given that the C-arm 28 is inoperative to slide over 90-degrees due to the design convenience even when it is preferred that the C-arm slides over 90-degrees (e.g., 100-degrees) from such a default state in the direction indicated by the arrow E in FIG. 9 to carry out the X-ray fluoroscopy or the X-ray imaging. In such a case, the C-arm 28 starts sliding following the operation of the lever 56 of the operation unit 3 by the operator referring to FIG. 5. In such a case, the control unit 4 sends the directive for sliding the C-arm 28 to the movement mechanism 53 of the X-ray imaging unit 2.

Referring to FIG. 10, the control unit 4 sends a signal to the movement mechanism 53 of the X-ray imaging unit 2 to suspend the slide of the C-arm 28 when the C-arm rotates 90-degrees. Then, the control unit 4 sends the directive to the tilting-and-rolling mechanism 51 of the examination table 1 to roll the table 11 of the examination table 1 toward the direction indicated by the arrow F in FIG. 10.

The operator suspends the operation of the lever 56 of the operation unit 3 when the angle θ between the axis line from the X-ray irradiation element 31 to the flat panel detector 32 and the surface of the table 11 coincides with the target angle, at which the X-ray fluoroscopy or the X-ray imaging is carried out, according to the rolling action of the table 11. Accordingly, the rolling action of the table 11 suspends.

In such a state, the angle θ between the axis line from the X-ray irradiation element 31 to the flat panel detector 32 and the surface of the table 11 coincides with the target angle at which the X-ray fluoroscopy or the X-ray imaging is carried out. However, the position for the X-ray fluoroscopy or the position for the X-ray imaging relative to the subject shifts along with the rolling action of the table 11 and in addition, the subject is closer to the X-ray irradiation element 31. Therefore, the table 11 is moved in the width direction indicated by the arrow G in FIG. 11 and lowered as indicated by the arrow D using the movement mechanism 52 of the examination table 1 so that the X-ray transmits the always same region of the subject and the distance between the subject and the flat panel detector 32 or the X-ray irradiation element 31 is constant.

In addition, in such a way, the C-arm 28 slides in the oblique direction as well as that the table 11 rolls, the shift amount is limited, so that the movements of the table 11 in the direction indicated by the arrow G and the arrow H can be skipped.

In addition, in the Embodiment set forth above, when the operator operates the lever 56 of the operation unit 3 and then the angle θ between the axis line from the X-ray irradiation element 31 to the flat panel detector 32 and the surface of the table 11 coincides with the target angle at which the X-ray fluoroscopy or the X-ray imaging is carried out, the slide of the C-arm 28 suspends. On the other hand, when the C-arm 28 slides to the position corresponding to the three-dimensional image by rotating the three-dimensional image denoting the C-arm 28 and using the three-dimensional image interlocking operation, the calculation element 54 of the control unit 4 calculates the sliding amount of the C-arm 28 and the tilting amount of the table 11 as well as the Embodiment set forth above.

Specifically, when the C-arm 28 slides in such a way, the operator inputs the target angle using the input element 57, such as a keyboard, of the operation unit 3, at which the X-ray fluoroscopy or the X-ray imaging is carried out, Then, the calculation element 54 of the control unit 4 calculates the angle of the rolling of the table 11 along with the slide of the C-arm 28 based on the locational information including such as the dimension and arrangement of each element of the examination table 1 and the X-ray imaging unit 2, which are stored in the memory storage 55, so that the imaging angle coincides with the target angle. And, based on the calculation results, the tilting-and-rolling mechanism 51 of the examination table 1 rolls the table 11 and in addition, the movement mechanism 53 of the X-ray imaging unit 2 slides the C-arm 28.

According to the Embodiment referring to FIG. 9-FIG. 11, the C-arm 28 approaches the subject from the side thereof and the C-arm 28 slides, so that the X-ray irradiation element 31 and the flat panel detector 32 move in the oblique direction relative to the subject. On the other hand, alternatively, the C-arm 28 approaches the subject from the head thereof and the C-arm 28 rotates in accordance with actions of the slide member 27 and the rotation member 29, so that the X-ray irradiation element 31 and the flat panel detector 32 move in the oblique direction relative to the subject. In such a case, the rolling action and the movement action of the table 11 are the same as the Embodiment set forth referring to FIG. 9-FIG. 11.

In addition, according to the two Embodiments set forth above, the C-arm 28 approaches the subject from either one side of the head thereof and the side of the body thereof, but the C-arm 28 may approach from the oblique direction thereof. In such a case, the X-ray irradiation element 31 and the flat panel detector 32 move in the combination direction of the sagittal direction and the oblique direction, so that the sliding action of the C-arm 28 and the rolling action thereof and the rolling action of the table 11 and the tilting action thereof can be executed in combination as needed.

### REFERENCE OF SIGNS

1 Examination table
2 X-ray Imaging unit
3 Operation unit
4 Control unit
11 Table
12 Frame
13 Pedestal
14 Axis
Axis
16 Movement table
17 Lifting pedestal
18 Support base
27 Sliding member
28 C-arm (arcuate arm)
29 Rotation member
31 X-ray irradiation element
32 Flat panel detector
51 Tilting-and-rolling mechanism
52 Movement mechanism
53 Movement mechanism
54 Calculation element
55 Memory Storage element
56 Lever
57 Input element

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Those of skill would further appreciate that the various illustrative logical blocks, modules, circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, elements, circuits, aspects, and circuits described in connection with the embodiments disclosed herein, may be implemented or performed with a general or specific purpose processor, or with hardware that carries out these functions, e.g., a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. The processor can be part of a computer system that also has an internal bus connecting to cards or other hardware, running based on a system BIOS or equivalent that contains startup and boot software, system memory which provides temporary storage for an operating system, drivers for the hardware and for application programs, disk interface which provides an interface between internal storage device(s) and the other hardware, an external peripheral controller which interfaces to external devices such as a backup storage device, and a network that connects to a hard wired network cable such as Ethernet or may be a wireless connection such as a RF link running under a wireless protocol such as 802.11. Likewise, an external bus may be any of but not limited to hard wired external busses such as IEEE-1394 or USB. The computer system can also have a user interface port that communicates with a user interface, and which receives commands entered by a user, and a video output that produces its output via any kind of video output format, e.g., VGA, DVI, HDMI, display port, or any other form. This may include laptop or desktop computers, and may also include portable computers, including cell phones, tablets such as the IPAD™ and Android™ platform tablet, and all other kinds of computers and computing platforms.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

The steps of a method or algorithm described in connection with the embodiments disclosed herein may be embodied directly in hardware, in a software module executed by a processor, using cloud computing, or in combinations. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of tangible storage medium that stores tangible, non-transitory computer-based instructions. An exemplary storage medium is coupled to the processor such that the processor can read information from, and write information to, the storage medium. In the alternative, the storage medium may be integral to the processor. The processor and the storage medium may reside in reconfigurable logic of any type.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

The memory storage can also be rotating magnetic hard disk drives, optical disk drives, or flash memory-based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media. The computer readable media can be an article comprising a machine-readable non-transitory tangible medium embodying information indicative of instructions that when performed by one or more machines result in computer implemented operations comprising the actions described throughout this specification.

Operations as described herein can be carried out on or over a web site. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

The computers described herein may be any kind of computer, either general purpose, or some specific purpose computer such as a workstation. The programs may be written in C, or Java, Brew or any other programming language. The programs may be resident on a storage medium, e.g., magnetic or optical, e.g. the computer hard drive, a removable disk or media such as a memory stick or SD media, or other removable medium. The programs may also be run over a network, for example, with a server or other machine sending signals to the local machine, which allows the local machine to carry out the operations described herein.

Also, the inventors intend that only those claims which use the words “means for” are intended to be interpreted under 35 USC 112, sixth paragraph. Moreover, no limitations from the specification are intended to be read into any claims, unless those limitations are expressly included in the claims.

It will be further understood by those of skill in the art that the arcuate type C-arm is an operative arm that has a generally arcuate shape with two generally related ends, but is not strictly a particular required arc-amount (e.g., 180 degrees, 190 degrees, 240 degrees, etc. Those of skill in the art of such radiation fluoroscopic design matters will recognize that related arcuate-type arms may be provided without departing from the scope and spirit of the present invention.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A radiation fluoroscopy imaging apparatus, comprising:
an examination table, that further comprises:
a table on which a subject is loaded, a tilting-and-rolling mechanism that carries out at least one action selected from a group consisting of a tilting action of said table and a rolling action of said table; and
a radiation imaging unit, that further comprising:
a radiation irradiation element;
a radiation detector that detects a radiation that is irradiated from said radiation irradiation element and transmits through said subject on said table;
a C-arm that supports said radiation irradiation element and said radiation detector that are facing each other; and
a C-arm movement mechanism that supports said C-arm slidably and rotatably;
wherein, said table tilting-and-rolling mechanism carries out at least one action selected from said group consisting of said tilting action of said table and said rolling action thereof so that an imaging angle coincides with a target angle when said target angle, at which an operator achieves at least one operation selected from a group consisting of a radiation fluoroscopy and a radiation imaging, is out of a range of said imaging angle along with at least one action selected from said group consisting of said tilting action of said C-arm and said rolling action thereof.

2. The radiation fluoroscopy imaging apparatus, according to claim 1, wherein:
said examination table, further comprises:
a table movement mechanism that is operative to move said table in a longitudinal direction, a width direction, and a vertical direction relative to said table; and
said movement mechanism moves said table in at least one direction selected from the group consisting of said longitudinal direction, said width direction and said vertical direction so that the radiation from said radiation irradiation element toward said radiation detector passes always through the same location relative to said subject and said distance between said subject and at least one element selected from the group consisting of said radiation irradiation element and said radiation detector is constant when said table tilting-and-rolling mechanism carries out at least one selected action from the group consisting of said tilting action and said rolling action relative to said table.

3. The radiation fluoroscopy imaging apparatus, according to claim 2, wherein:
when said radiation irradiation element and said radiation detector move in the sagittal direction relative to said subject, said table tilting-and-rolling mechanism tilts said table and said table movement mechanism moves said table in said longitudinal direction and said vertical direction.

4. The radiation fluoroscopy imaging apparatus, according to claim 2, wherein:
when said radiation irradiation element and said radiation detector move in the oblique direction relative to said subject, said table tilting-and-rolling mechanism rolls said table and said table movement mechanism moves said table in said width direction and said vertical direction.

5. The radiation fluoroscopy imaging apparatus, according to claim 1, wherein:
said table carries out at least one action selected from the group consisting of said tilting action and said rolling action after said C-arm slides nearby a limited sliding amount when said C-arm slides and said C-arm rotates nearby a limited rotation amount when said C-arm rotates.

6. The radiation fluoroscopy imaging apparatus, according to claim 1 further comprising:
an input element that is operative to input said target angle; and
a calculation element that calculates a required angle for at least one action selected from the group consisting of said tilting action of said table and said rolling action thereof from said target angle and an imaging angle in accordance with at least one action selected from the group consisting of said sliding action and said rotation action of said C-arm; and
wherein said table tilting-and-rolling mechanism is operative to carry out at least one action selected from the group consisting of said tilting action of said table and said rolling action thereof based on the calculation results provided by said calculation element.
